(19) United States
(12) Patent Application Publication
(10) Pub. No.: US 2026/0115196 A1
(43) Pub. Date: Apr. 30, 2026

(54) DIRECT DELIVERY OF ANTIOXIDANTS TO THE GUT

(71) Applicant: DSM IP Assets B.V., Maastricht (NL)

(72) Inventors: Thanh-Van PHAM, Kaiseraugst (CH); Ateequr REHMAN, Kaiseraugst (CH); Robert STEINERT, Kaiseraugst (CH); Wilbert SYBESMA, Kaiseraugst (CH)

(21) Appl. No.: 19/429,458

(22) Filed: Dec. 22, 2025

Related U.S. Application Data
(62) Division of application No. 17/798,778.

(30) Foreign Application Priority Data
Feb. 12, 2020 (EP) 20156790.6

(51) Int. Cl. A61K 31/525; A61K 31/355; A61K 31/375; A61K 45/06; A61P 1/14; A61P 3/02

(52) U.S. Cl. CPC A61K 31/525; A61K 31/355; A61K 31/375; A61K 45/06; A61P 1/14; A61P 3/02

(57) ABSTRACT

A combination of antioxidants which are delivered to the gut microbiome provides a degree of improbment to gut health comparable to known prebiotics, such as soluble fibers. The antioxidants are riboflavin, beta-carotene, Vitamin C and Vitamin E.

### CROSS-REFERENCE TO RELATED APPLICATIONS

This application is a divisional of U.S. application Ser. No. 17/798,778 filed Aug. 10, 2022 (now abandoned), which in turn is the U.S. national phase of International Application No. PCT/EP2020/083016 filed Nov. 23, 2020 which designated the U.S. and claims priority to EP Patent Application No. 19211703.4 filed Nov. 27, 2019, the entire contents of each of which are hereby incorporated by reference.

### BRIEF DESCRIPTION OF THE INVENTION

This invention relates to the direct delivery of a composition comprising at least two antioxidants such as Vitamin C, Vitamin B2, beta-carotene and Vitamin E to the gut microbiome. The composition was found to have microbiome-modulating/prebiotic activity, resulting in increased short chain fatty acid production, an increased abundance of beneficial bacterial, and increased activity of the gut microbiome.

### BACKGROUND OF THE INVENTION

Direct delivery of various vitamins and other active ingredients to the gut has been described. See, e.g. U.S. Pat. No. 9,433,583 B2 directed to a colon-targeted single dosage form comprising vitamin D and optionally further vitamins for preventing colorectal adenomatous polyps and colorectal cancer and WO2014/070014 directed to the use of riboflavin (Vitamin B2) to stimulate the population of Faecalibacterium prausnitzii.

It would be desirable to have a combination of active ingredients which act in a synergistic manner to improve gut microbial activity.

### DETAILED DESCRIPTION OF THE INVENTION

It has been found, in accordance with this invention that combinations of antioxidants, specifically the combination of Vitamin C, Vitamin B2, Vitamin E and beta-carotene, when delivered directly to the gut, can have a synergistic microbiome modulating effect on the gut microbiome. Thus one aspect of this invention is a method of enhancing gut health comprising administering a combination of antioxidants directly to the gut.

Preferably the antioxidants are at least two, and more preferably all of: Vitamin C, Vitamin E, riboflavin and beta-carotene.

Thus, one embodiment of this invention is a composition consisting essentially of:
an effective dose of at least two antioxidants selected from the group consisting of: Vitamin C, Vitamin B2, beta-carotene and Vitamin E
for use in improving intestinal health in an animal, including a human, wherein said improvement comprises or consists of:
   i. Increasing the activity of the microbiome;
   ii. increasing the concentration of at least one short-chain fatty acid or a salt thereof in the intestine;
   iii. reducing the amount of ammonia formed in the gut;
   iv. increasing microbiome diversity in the intestine;
   v. increasing the abundance of a beneficial bacteria in the intestine;
   vi. improving the barrier function of the intestine; and/or
   vii. decreasing the abundance of pathogens in the intestine;
said composition being delivering the antioxidants to the large intestine.

Another embodiment of this invention is the use of
an effective dose consisting essentially of at least two antioxidants selected from the group consisting of: Vitamin C, Vitamin B2, beta-carotene and Vitamin E for use in improving intestinal health in an animal, including a human, wherein said improvement comprises or consists of:
   i. Increasing the activity of the microbiome
   ii. increasing the concentration of at least one short-chain fatty acid or a salt thereof in the intestine;
   iii. reducing the amount of ammonia formed in the gut;
   iv. increasing microbiome diversity in the intestine;
   v. increasing the abundance of a beneficial bacteria in the intestine;
   vi. improving the barrier function of the intestine; and/or
   vii. decreasing the abundance of pathogens in the intestine;
      in the manufacture of a medicament or nutraceutical which delivers the antioxidants to the large intestine.

Another embodiment of this invention is a method of improving intestinal health in an animal, including a human, comprising administering to the animal a composition selected from the group consisting essentially of:
an effective dose of at least two antioxidants selected from the group consisting of: Vitamin C, Vitamin B2, beta-carotene and Vitamin E;
wherein said improvement comprises or consists of:
   i. Increasing the activity of the microbiome
   ii. increasing the concentration of at least one short-chain fatty acid or a salt thereof in the intestine;
   iii. reducing the amount of ammonia formed in the gut;
   iv. increasing microbiome diversity in the intestine;
   v. increasing the abundance of a beneficial bacteria in the intestine;
   vi. improving the barrier function of the intestine; and/or
   vii. decreasing the abundance of pathogens in the intestine;
      said composition delivering the antioxidants to the large intestine. In some embodiments, the animal is in need of such antioxidants.

In preferred embodiments, the antioxidants comprise all four antioxidants.

### BRIEF DESCRIPTION OF THE FIGURES

FIG. 1 shows the modified version of a continuous batch fermentation model (such as the SHIME, or TWINSHIME, or QuadSHIME provided by Prodigest, Technologiepark-Zwijnaarde 94, 9052 Gent, Belgium) which were used for the current study. St: Stomach vessel, SI: Small Intestine vessel, St/SI: vessel serving as stomach and small intestine, PC: Proximal colon and DC: Distal colon.

FIG. 2. Effect of the DSM non-prebiotic blend (ANTIOX), and the commercial prebiotic (FOS) and (XOS) treatments compared to a blank control (CTRL). FIG. 2A shows acetate production (mM) in the proximal (PC) for donor B; FIG. 2B shows butyrate production (mM) in the proximal (B) and distal colon (C) for donor A.

FIG. 3. Effect of the DSM non-prebiotic blend (ANTIOX), and the commercial prebiotic (FOS) and (XOS) treatments compared to a blank control (CTRL) on the abundance of Bifidobacterium longum in proximal (A and C) and distal colon (B and D) for donor A (upper panel) and donor B (lower panel).

FIG. 4. Effect of the DSM non-prebiotic blend (ANTIOX), and the commercial prebiotic (FOS) and (XOS) treatments compared to a blank control (CTRL) on the abundance of Bacterides fragilis in the proximal (A) and distal colon (B) for donor A.

### DEFINITIONS

As used throughout, the following definitions apply:

The term “riboflavin” which can be used interchangeably with “Vitamin B2”, includes riboflavin and esters thereof, in particular riboflavin-5′-phosphate.

The term “vitamin C” which can be used interchangeably with “ascorbic acid” also includes pharmaceutically acceptable salts thereof (e.g. sodium ascorbate and calcium ascorbate) and pharmaceutically acceptable esters thereof (in particular ascorbyl palmitate).

The term “β-carotene” refers to β-carotene or Provitamin A.

The term “vitmain E” includes four forms of tocopherols (alpha-Tocopherol, beta-Tocopherol, gamma-Tocopherol and delta-Tocopherol) and four forms of tocotrienols (alpha-tototrinols, beta-tocotrienols, gamma-tocotrienols and delta-tocotrienols)

The term “short-chain fatty acid” (SCFA) as used herein refers to fatty acids with two to six carbon atoms. SCFAs include formic acid, acetic acid, propionic acid, butyric acid, 2-methylpropanoic acid, 3-methylbutanoic acid, and hexanoic acid. The most important SCFAs are acetic acid, propionic acid and butyric acid.

“Increasing Short Chain Fatty Acid (SCFA) production” includes increasing any or all of: acetic acid, propionic acid, and butyric acid production) as well as increasing lactate production, as lactate is a SCFA precursor. “Increasing SCFA” can also include decreasing ammonium and branched SCFAs (isobutyric acid, isovaleric acid and isocaptoic acid, which are markers of proteolytic fermentation, with generally adverse effects on host health).

“Increase in microbiome activity” includes increased base consumption, also includes increasing overall SCFA production, increase in any or all of acetic acid, propionic acid, and butyric acid production as well as increasing lactate production. Decreased microbiome activity such as reduced intestinal SCFA production correlates with diseases including obesity and inflammatory bowel disease.

### Increase in Short Chain Fatty Acids

The “increase” in the concentration of one or more SCFA in the colon of an animal is relative to the SCFA concentration of an animal not administered with the active antioxidants. The animal not administered the active antioxidants is referred to herein as “control animal”. Preferably, the increase in the concentration of one or more SCFA in the colon of an animal is relative to the SCFA concentration of the same animal not administered the active antioxidants. That is, the control animal in this situation is the same animal typically prior to the start of the administration of the active antioxidants. Preferably, the control animal has not received an active antioxidants recited herein in the form of nutritional supplements for at least 28 days.

In one embodiment, the concentration of one or more SCFAs is increased upon a single administration of the active antioxidants. In another embodiment, the concentration of one or more SCFAs is increased upon two administrations of the active antioxidants, which are applied on two consecutive days. In another embodiment, the concentration of one or more SCFAs is increased upon seven administrations of the active antioxidants, which are applied on seven consecutive days. In another embodiment, the concentration of one or more SCFAs is increased upon 14 administrations of the active antioxidants, which are applied on 14 consecutive days. Preferably, the concentration of one or more SCFAs (e.g., of acetic acid, propionic acid and/or butyric acid) is increased upon 21 administrations of the active antioxidants, which are applied on 21 consecutive days. Most preferably, the concentration of one or more SCFAs (e.g., of acetic acid, propionic acid and/or butyric acid) is increased upon 28 administrations of the active antioxidants, which are applied on 28 consecutive days, once daily. The concentration of the SCFA in the colon may be increased by at least 5%, preferably by at least 10%, or at least 15%, or at least 20%, relative to the SCFA concentration in the colon of a control. In the control no active antioxidants has been administered.

The concentration of SCFAs in the colon can be determined by obtaining fecal samples from the mammal administered with the active antioxidants and measuring the concentration of one or more SCFAs.

Methods for measuring the concentration of commonly known SCFAs, e.g. by gas chromatography, are known to those of skill in the art. For example, De Weirdt et al (2010) (DOI:10.1111/j.1574-6941.2010.00974.x) describes suitable methods.

Alternatively, the increase of one or more SCFAs upon administration of the active antioxidants can be determined using a reactor and fecal suspension as an in vitro model, as described in the Examples of this application.

It was also observed that an increase in SCFAs was accompanied by a decrease in the abundance of pathogenic bactieria such as Bacteroides fragilis.

Another embodiment of this invention is use of at least two and preferably four antioxidants selected from the group consisting of Vitamin C, Vitamin B2, Vitamin E and beta-carotene, in the improvement of intestinal health in an animal, including a human, wherein the improvement comprises increasing the concentration of at least one short-chain fatty acid or a salt thereof in the intestine; and wherein the short-chain fatty acid is selected from the group consisting of: of acetic acid, propionic acid and butyric acid or salts thereof.

Another embodiment of this invention an active antioxidant composition wherein the active antioxidant is at least two selected from the group consisting of: beta carotene, Vitamin C, Vitamin E and riboflavin for use in increasing the concentration of at least one short-chain fatty acid or a salt thereof in the intestine.

Another embodiment of this invention is an active antioxidant composition for use in the improvement of intestinal health in an animal, including a human, wherein the improvement comprises increasing the concentration of at least one short-chain fatty acid or a salt thereof in the intestine; wherein the animal, including a human, is experiencing a condition selected from the group consisting of: metabolic disorder, Type 2 Diabetes, obesity, Crohn's disease, ulcerative colitis, inflammatory bowel disease, irritable bowel syndrome, leaky gut, malnutrition, chronic inflammation, and cardiovascular disease.

### Improving Diversity of the Microbiome

Another embodiment of this invention is the use of the antioxidant composition to increase the diversity of the microbiome, and/or increase the amount of beneficial bacteria of the intestine, and specifically in the colon. The beneficial bacteria which are known to inhabit the colon include Acidaminococcus, Akkermansia sp. Bacteroides ovatus, Bifidobacterium spp., Blautia producta, Clostridium cocleatum, Collinsella aerofaciens, Dorea longicatena, Escherichia coli, Eubacterium spp., Faecalibacterium prausnitzii, Lachnospira pectinoshiza, Lactobacillus spp., Parabacteroides distasonis, Raoultella spp., Roseburia spp., Ruminococcus spp., and Streptococcus spp.

Preferably the bacteria which are increased are selected from the group consisting of Bifidobacterium, Akkermansia, Faecalibacterium and Bacteroides. More preferably Bifidobacterium adolescentis, Bifidobacterium longum, Bacteroides ovatus, Bacteroides xylanisolvens, Lachnoclostridium sp. Akkermansia muciniphila, Blautia wexlerae, and/or Faecalibacterium prausnitzii are increased after administration of the antioxidants of this invention.

Increasing the diversity of bacteria and/or increasing the amount of beneficial bacteria is particularly helpful when the animal, including a human, is experiencing a condition selected from the group consisting of: metabolic disorder, Type 2 Diabetes, obesity, Crohn's disease, ulcerative colitis, inflammatory bowel disease, irritable bowel syndrome, leaky gut, malnutrition, chronic inflammation, and cardiovascular disease.

### Improving the Barrier Function of the Intestine

Another embodiment of this invention is the use of the antioxidant composition to increase the barrier function of the intestine. Improvement of the barrier function is particularly important when the animal, including the human, is experiencing a condition where the barrier function is impaired, such as a condition selected from the group consisting of: metabolic disorder, Type 2 Diabetes, obesity, Crohn's disease, ulcerative colitis, inflammatory bowel disease, irritable bowel syndrome, leaky gut, malnutrition, chronic inflammation, and a cardiovascular disease. SCFA act as a fuel for intestinal epithelial cells and are known to support the gut barrier function, butyrate specially have immunomodulatory effect.

### Doses:

Preferably, riboflavin is administered in an amount such that its local concentration in the colon is at least 0.05 g/L, preferably at least 0.1 g/L more preferably at 0.125 g/L. Preferred local concentrations in the colon range from about 0.1 g/L to about 0.5 g/L or from about 0.1 g/L to about 0.2 g/L, preferably about 0.125 g/L. One preferred dosage per day can be up to 200 mg.

Preferably, β-carotene is administered in an amount such that its local concentration in the colon is at least 0.1 g/L, preferably at least 0.15 g/L, most preferably at least 0.2 g/L. Preferred local concentrations in the colon range from about 0.05 g/L to about 0.4 g/L, more preferably from about 0.15 g/L to about 0.25 g/L One preferred dosage per day is up to 150 mg.

Preferably, vitamin E (50%) is administered in an amount such that its local concentration in the colon is at least 0.005 g/L preferably at least 0.05 g/L, most preferably at least 0.15 g/L. Preferred local concentrations in the colon range from about 0.005 g/L to about 2.5 g/L, more preferably from about 0.15 g/L to about 1.75 g/L. One preferred dosage per day is up to 1000 mg.

Preferably, ascorbic acid is administered in an amount such that its local concentration in the colon is at least 0.05 g/L, preferably at least 0.1 g/L, most preferably at least 0.8 g/L. Preferred local concentrations in the colon range from about 0.05 g/L to about 1.5 g/L, more preferably from about 0.5 g/L to about 1 g/L, most preferably from about 0.8 g/L to about 0.9 g/L. One preferred dosage per day is up to 2000 mg.

Preferably the antioxidants are present in a ratio of:

| | | |
|---|---|---|
| Riboflavin | 0.5 | to 2 |
| Acorbic Acid | 4 | to 15 |
| Vitamin E | 1 | to 5 |

| | |
|---|---|
| Beta-Carotene | 0.5-3 |

More preferably the ratio of Riboflavin/Ascorbic acid/Vitamin E/β-Carotene is 1.0/6.6/1.3/1.6.

In preferred embodiments, the compositions are administered for an extended period time, such as for at least once per day for at least 3 days, at least a week, at least two weeks and at least 4 weeks.

The antioxidants are preferably administered in a formulation which allows the actioxidant to be released in the intestine. Such forms are know in the art. Alternatively, and perhaps preferably for non-human administration, the animal is administered a high enough dose for the anioxidant to be present in the instestine.

The following non-limiting Examples are presented to better illustrate the invention

### EXAMPLES

The aim of this study was to compare the effect of directly delivered antioxidants to that of two established prebiotics: Xylooligosacchrides (XOS) and Fructooligosaccharides (FOS).

Two donors were selected for the long-term SHIME® experiment, where the impact of repeated intake of the test products was evaluated on the activity (as assessed via SCFA, lactate, branched SCFA and ammonia production) and composition (as assessed via 16S Illumina sequencing) of the luminal gut microbiome.

### Design of the SHIME® Experiment

The typical reactor setup of the SHIME® represents the gastrointestinal tract of the adult human. It has a succession of five reactors simulating the different parts of the human gastrointestinal tract. The first two reactors are of the fill-and-draw principle to simulate different steps in food uptake and digestion, with peristaltic pumps adding a defined amount of SHIME feed (140 mL 3×/day) and pancreatic and bile liquid (60 mL 3×/day), respectively to the stomach (V1) and small intestine (V2) compartment and emptying the respective reactors after specified intervals. The last three compartments simulate the large intestine. These reactors are continuously stirred; they have a constant volume and pH control. Retention time and pH of the different vessels are chosen to resemble in vivo conditions in the different parts of the colon. Upon inoculation with fecal microbiota, these reactors simulate the ascending (V3), transverse (V4) and descending (V5) colon. Inoculum preparation, retention time, pH, temperature settings and reactor feed composition have been described elsewhere. Upon stabilization of the microbial community in the different regions of the colon, a representative microbial community is established in the three colon compartments, which differs both in composition and functionality in the different colon regions.

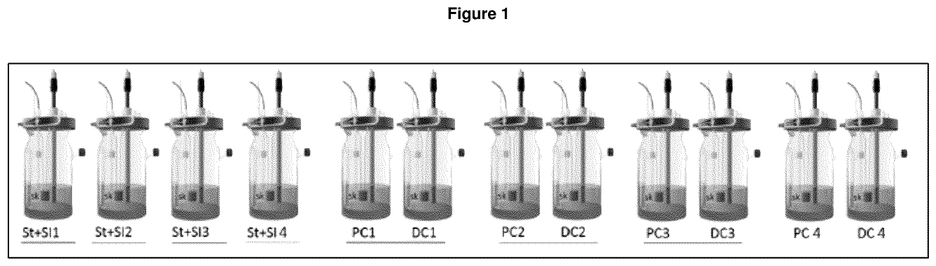

The conventional SHIME setup was adapted from a TWINSHIME configuration to a QuadSHIME® configuration (FIG. 1) allowing to compare four different conditions in parallel. During this specific project, the properties of three different test ingredients and a blank control were evaluated in two parallel TripleSHIME® configurations using the microbiota of two healthy adult human donors, meaning that each donor was tested in a separate QuadSHIME® experiment. As a compromise for the additional test conditions, the colon regions were limited to two regions as compared to three regions in the TWINSHIME. The retention times and pH ranges were optimized in order to obtain results that are representative of a full GIT simulation. In practice, in QuadSHIME® experiments, instead of working with 2 units, each composed of an AC-TC-DC configuration (ascending, transverse and descending colon), one used 4 PC-DC units. Upon inoculation with a faecal microbiota of a human adult, these reactors simulate the proximal colon (PC; pH 5.6-5.9; retention time=20 h; volume of 500 mL) and distal colon (DC; pH 6.6-6.9; retention time=32 h; volume of 800 mL).

The SHIME® experiment for this study consisted of two stages (Table 1, below): Stabilization period: After the inoculation of the colon reactors with an appropriate fecal sample, a two-week stabilization period allowed the microbial community to differentiate in the different reactors depending on the local environmental conditions. During this period the basic nutritional matrix was provided to the SHIME to support the maximum diversity of the gut microbiota originally present in the fecal inoculum. Analysis of samples at the end of this period allows to determine the baseline microbial community composition and activity in the different reactors.

Treatment period: During this two-week period, the SHIME reactor was operated under nominal conditions, but with a diet supplemented with the test product. Samples taken from the colon reactors in this period allow to investigate the specific effect on the resident microbial community composition and activity. For the blank control condition, the standard SHIME nutrient matrix was further dosed to the model for a period of 14 days. Analysis of samples of these reactors allow to determine the nominal microbial community composition and activity in the different reactors, which will be used as a reference for evaluating the treatment effects.

TABLE 1
Overview of the different stages applied in this study.

| Week 1 | Week 2 | Week 3 | Week 4 |
|---|---|---|---|
| Stabilization | Stabilization | Treatment | Treatment |

Samples were collected at the following time points to follow up on the adaptation of the microbiota to the different test products:
Last three days of stabilization period;
Last two days of the first treatment week;
Last two days of the second treatment week.

### Analysis of the Microbial Community Composition and Activity

An important characteristic of the SHIME is the possibility to work with a stabilized microbiota community and to regularly collect samples from the different intestinal regions for further analysis. The large volumes in the colonic regions allow to collect sufficient volumes of liquids each day, without disturbing the microbial community or endangering the rest of the experiment. A number of microbial parameters are monitored throughout the entire SHIME experiment. These measurements are necessary to evaluate the performance of the model and allow to monitor basic changes in the microbial community composition and activity due to the prebiotic treatment.

### Overall Fermentative Activity:

Acid/base consumption: the production of microbial metabolites in the colon reactors alters the pH. Without continuous pH control (through the addition of acid or base), the pH would exceed the fixed intervals. Consumption of acid/base is continuously monitored.

### Microbial Community Activity:

Short-chain fatty acids (SCFA): the concentrations of acetic acid, propionic acid, and butyric acid were analyzed.

Lactate: precursor of SCFA and potential antimicrobial agent.

Ammonium and branched SCFA (isobutyric acid, isovaleric acid, and isocaproic acid) are markers of proteolytic fermentation, with rather adverse effects on host health.

Microbial Community Composition: Samples were collected for 16S-targeted Illumina sequencing.

### Analysis of the Microbial Community Composition

Two techniques were combined to map the community shifts induced by the different treatments in large detail:
16S-targeted Illumina sequencing, a PCR-based method by which microbial sequences are amplified until saturation, thus providing proportional abundances of different taxa at different phylogenetic levels (microbial phylum, family and OTU level). The methodology applied by ProDigest involves primers that span 2 hypervariable regions (V3-V4) of the 16S rDNA. Using a paired sequencing approach, sequencing of 2×250 bp results in 424 bp amplicons. Such fragments are taxonomically more useful as compared to smaller fragments that are taxonomically less informative.
Accurate quantification of total bacterial cells in the samples through flow cytometry. Combining the high-resolution phylogenetic information of the 16S-targeted Illumina together with the accurate enumeration of the cell count via flow cytometry, highly accurate, quantitative abundances of the different taxonomic entities inside the reactors can be obtained.

Additional samples were taken from each reactor, which were aliquoted and centrifuged at 10,000 rpm for 10 minutes at 4° C., followed by filtration through a 0.2 μm filter. Supernatants and pellets were shipped to DSM. Furthermore, 100 mL of SHIME nutritional medium and 100 mL of pancreatic juice were collected and shipped to DSM.

The comparisons of normally distributed data of the different stabilization and treatment weeks on microbial metabolic markers and microbial community parameters were performed with a Student's T-test assuming equal variance. Differences were considered significant if p<0.05.

### Trial

Three different test products were tested in this project as compared to a blank control. The test products and the in vitro doses at which they were tested can be found in Table 2.

TABLE 2
List of test products and the in vitro dosage at which
they were tested in the long-term SHIME experiment.

| | | | In vitro |
|---|---|---|---|
| | | Product | dosage (mg/d) |
| Blend 1 | Non-prebiotic | Riboflavin | 75 |
| | vitamin | Ascorbic acid | 500 |
| | antioxidant | Dry Vitamin E (50% form) | 200 |
| | mix (ANTIOX) | β-Carotene (10% form) | 1200 |
| Blend 2 | Prebiotic 1 | XOS | 3000 |
| Blend 3 | Prebiotic 2 | FOS | 3000 |

### Results

As required at the end of the stabilization period, acid/base consumption, SCFA, lactate, ammonium, and microbiota composition were all very stable within and reproducible between each of the SHIME units. This indicated that the SHIME model was operated under its most optimal conditions resulting in a stable and reproducible colon microbiota. This stability is a prerequisite to make firm statements that effects observed during the treatment truly result from the administered test products, while the high reproducibility allows for the direct comparison between the different test products.

### Microbial Activity Results

ANTIOX supplementation significantly increased base consumption in both colon regions for all donors tested when compared to control In the proximal colon, the strongest acidification was observed upon treatment with XOS. Interestingly, in donor B, the acid-base consumption in ANTIOX and FOS treatment groups were comparable to each other, suggesting that tested ANTIOX composition acts similar to established prebiotic FOS. In the distal colon, the strongest acid-base consumption was observed upon treatment with FOS in both tested donors. Interestingly, in donor A, microbial activity (acid-base consumption) in ANTIOX and XOS treatment were comparable suggesting that tested ANTIOX composition acts similar to established prebiotic XOS.

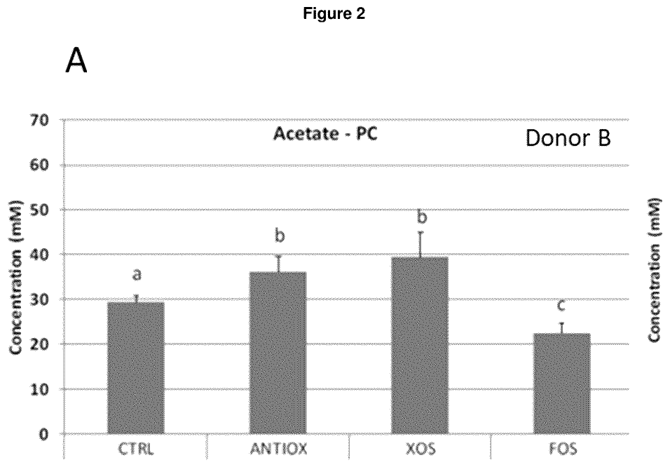

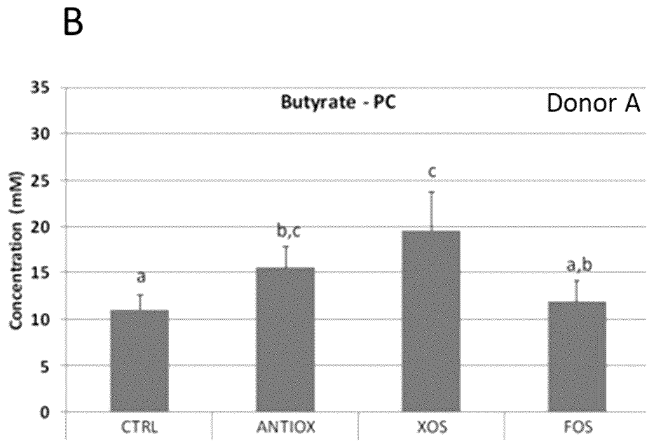

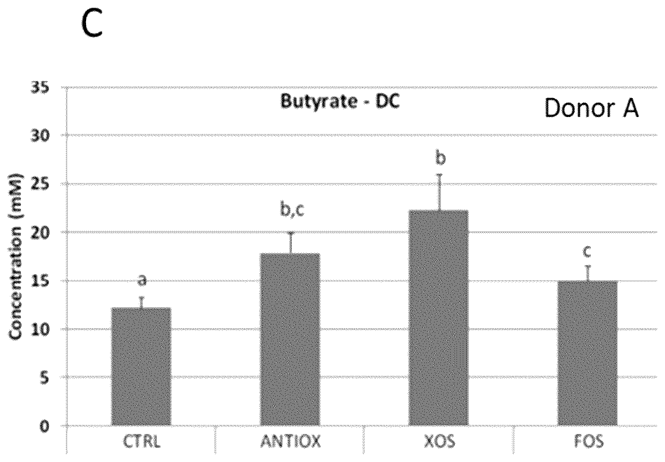

### Short-Chain Fatty Acid Results (FIG. 2)

As seen in FIG. 2, interestingly, the non-prebiotic vitamin antioxidant mix increased acetate and butyrate, but not propionate levels as compared to the control incubation. For donor A, increased acetate concentration was observed in antioxidant mix treated proximal colon vessels if compared to control as well as prebiotic FOS treated vessels. For donor B, increased SCFA butyrate was observed in proximal as well as in distal colon vessels if compared to control and prebiotic FOS treated vessels.

### Total SCFA Production

A similar effect was seen with total SCFA. The non-prebiotic antioxidant mix increased total SCFAs as compared to the control incubation with the magnitude of effect being similarly to the effects seen with well-established prebiotics

### Lactate

Likewise, In the proximal colon, a significant increase in lactate levels was observed upon supplementation of the ANTIOX and XOS blends as compared to the control incubation for both donors tested.

### Ammonium and Branched SCFA

The addition of the antioxidant blend resulted in decreased ammonium levels in both colon regions (proximal and distal colon), though less pronounced as the prebiotic test products. In the same line, administration of the antioxidant blend led to reduced branched SCFA production in proximal colon of donor A compared to control.

### Microbiome

1. Diversity
Treatment with ANTIOX LED to Increased Diversity in the Distal Colon and Proximal Colon.

Interestingly, in the proximal colon significantly reduced diversity was observed at the end of the treatment period upon treatment with the prebiotic blend 2 (XOS) and prebiotic blend 3 (FOS) as compared to the blank control.

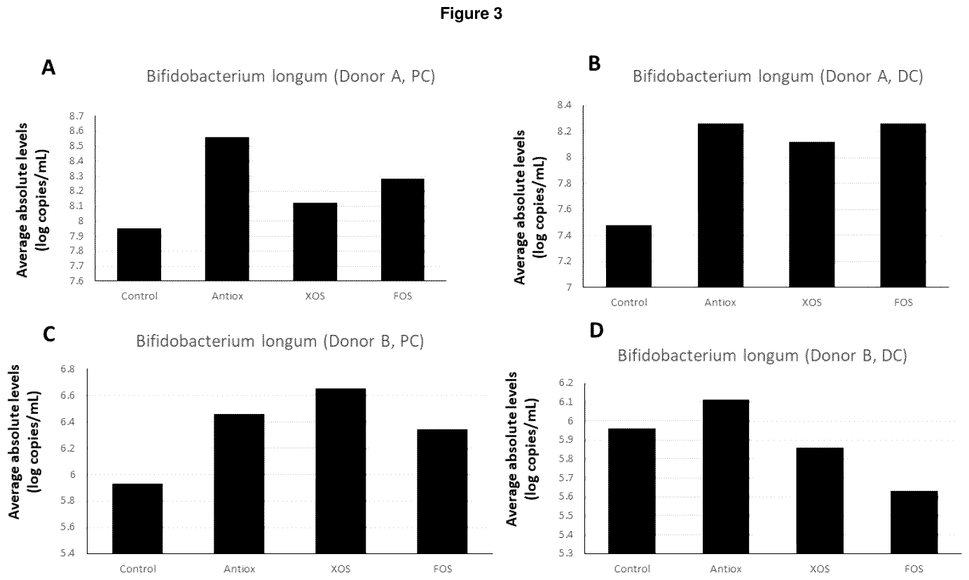

### 2. Increased Abundance of Beneficial Bacteria (FIG. 3)

Treatment of proximal and distal colon vessels with antioxidant resulted in increased Bifidobacterium longum abundance if compared to control. This observation was consistent for donor A and donor B. interestingly, this increase was even more than the prebiotic XOS and FOS treated proximal colon vessels for donor A. For donor A, distal colon, abundance of Bifidobacterium longum in antiox treated vessels was higher if compared to XOS. For donor B, abundance of Bifidobacterium longum in antiox treated vessels was higher than FOS treated proximal colon vessels while abundance of Bifidobacterium longum in antiox treated distal colon vessels was higher than the prebiotic XOS and FOS treated vessels.

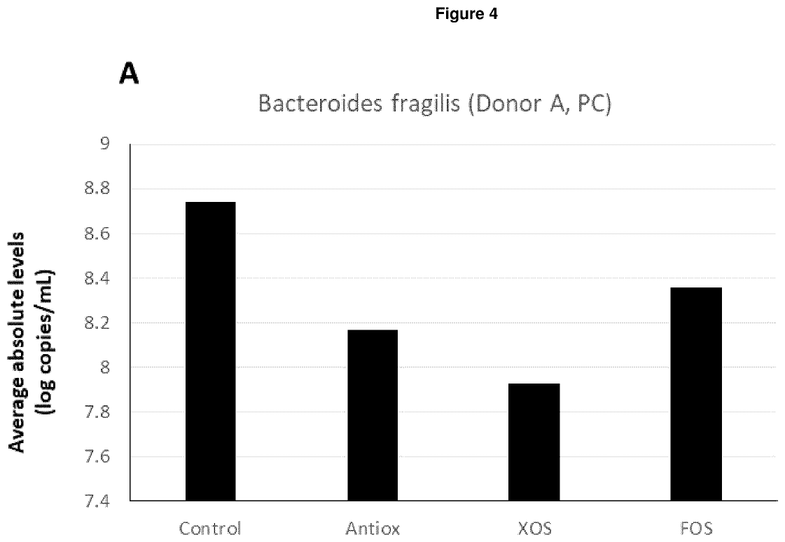

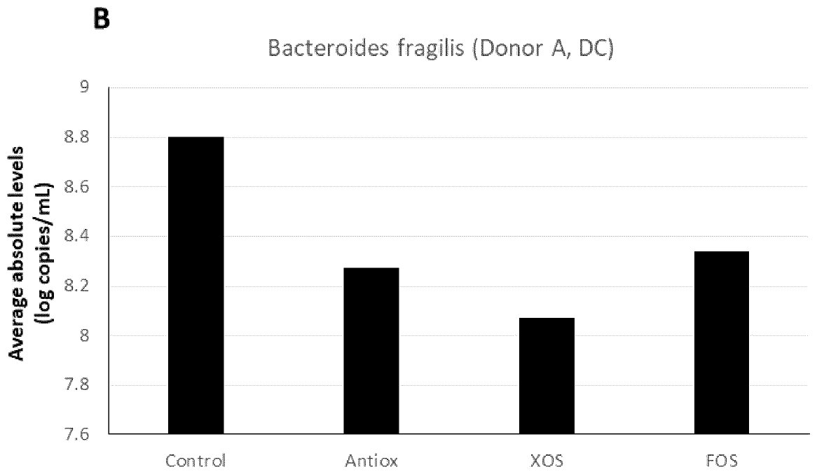

### 3. Decresaed Pathogenic Bacteria (FIG. 4)

For donor A, decrease in the abundance of opportunistic pathogen Bacterides fragilis was observed in antioxidant mix treated vessels if compared to control proximal and distal colon. Interestingly, this reduction in abundance was more than the prebiotic FOS.

1. A method of improving intestinal health in an animal, including a human, comprising administering to the animal a composition selected from the group consisting essentially of:
an effective dose of at least two antioxidants selected from the group consisting of Vitamin C, Vitamin B2, beta-carotene and Vitamin E;
wherein the intestinal health improvement comprises or consists of:
(i) increasing the activity of the microbiome
(ii) increasing the concentration of at least one short-chain fatty acid or a salt thereof in the intestine;
(iii) reducing the amount of ammonia formed in the gut;
(iv) increasing microbiome diversity in the intestine;
(v) increasing the abundance of a beneficial bacteria in the intestine;
(vi) improving the barrier function of the intestine; and/or
(v) decreasing the abundance of pathogens in the intestine;
wherein the composition delivers the antioxidants to the large intestine.

2. The method according to claim 1, wherein the antioxidant is Vitamin C, Vitamin E, Vitamin B2 and beta carotene.

3. The method according to claim 1, wherein the improvement is increasing at least one short-chain fatty acid and/or increasing the butyrate synthesis pathway activity in an animal.

4. The method according to claim 1, wherein the improvement is increasing a concentration of at least one short-chain fatty acid or a salt thereof in the intestine; and wherein the short-chain fatty acid is selected from the group consisting of of acetic acid, propionic acid and butyric acid or salts thereof.

5. The method according to claim 1, wherein the animal is experiencing a condition selected from the group consisting of: metabolic disorder, Type 2 Diabetes, obesity, Crohn's disease, ulcerative colitis, inflammatory bowel disease, irritable bowel syndrome, leaky gut, malnutrition, chronic inflammation, and cardiovascular disease.

6. The method according to claim 1, wherein the improvement is increasing microbiome diversity in the intestine, and/or increasing the abundance of a beneficial bacteria in the intestine.

7. The method according to claim 6, wherein the bacteria which are increased are selected from the group consisting of Bifidobacterium, Akkermansia, Faecalibacterium and Bacteroides.

8. The method according to claim 1, wherein the improving microbiome diversity is a method of treating, preventing or lessening a symptom of a condition selected from the group consisting of: metabolic disorder, Type 2 Diabetes, obesity, Crohn's disease, ulcerative colitis, inflammatory bowel disease, irritable bowel syndrome, leaky gut, malnutrition, chronic inflammation, and a cardiovascular disease.

9. The method according to claim 1, wherein the improvement is improving gut barrier function.

10. The method according to claim 1, wherein the improving the barrier function is a method of of treating, preventing or lessening a symptom of a condition selected from the group consisting of: metabolic disorder, Type 2 Diabetes, obesity, Crohn's disease, ulcerative colitis, inflammatory bowel disease, irritable bowel syndrome, leaky gut, malnutrition, chronic inflammation, and cardiovascular disease.

11. The method according to claim 1, wherein the animal is a human and the effective dose of the at least two antioxidants is delivered by a delayed release formulation.
